(19) United States
(12) Patent Application Publication
(10) Pub. No.: US 2003/0225067 A1
(43) Pub. Date: Dec. 4, 2003

(54) Microdialysis methods and applications for treatment and/or prophylaxis of tumors and/or infections in the central nervous system (CNS) and/or in other parenchymal organs

(76) Inventors: Ruediger Stendel, Berlin (DE); Rolf W. Pfirrmann, Lucerne (CH)

Correspondence Address: ROTHWELL, FIGG, ERNST & MANBECK, P.C., 1425 K STREET, N.W., SUITE 800, WASHINGTON, DC, 20005, US

(21) Appl. No.: 10/392,299

(22) Filed: Mar. 20, 2003

Related U.S. Application Data
(63) Continuation-in-part of application No. 10/281,138, filed on Oct. 28, 2002.
(62) Division of application No. 09/583,902, filed on Jun. 1, 2000, now Pat. No. 6,479,481.
(60) Provisional application No. 60/365,828, filed on Mar. 21, 2002.
(60) Provisional application No. 60/137,421, filed on Jun. 4, 1999.
(60) Provisional application No. 60/151,050, filed on Aug. 27, 1999.
(60) Provisional application No. 60/167,681, filed on Nov. 29, 1999.
(60) Provisional application No. 60/174,607, filed on Jan. 5, 2000.
(60) Provisional application No. 60/182,200, filed on Feb. 14, 2000.

(51) Int. Cl. A61K 31/54

(52) U.S. Cl. 514/222.5; 604/9

(57) ABSTRACT

The present invention relates, e.g., to a method and application for the treatment and/or prophylaxis of tumors and/or infections in the central nervous system and/or other parenchymal organs in mammalian subjects. In preferred embodiments, a microdialysis device is used to provide an effective dose of taurolidine and/or its metabolites and/or other chemotherapeutic and/or immunotherapeutic and/or antibiotic and/or virostatic and/or fungistatic agents is administed to a mammalian subject suffering from or at risk to growth of tumors of the central nervous system and/or other parenchymal organs.

### CROSS-RELATED REFERENCE TO RELATED APPLICATIONS

This application claims the benefit of U.S. Provisional Application No. 60/365,828, filed on Mar. 21, 2002 and is a continuation-in-part of U.S. application Ser. No. 10/281,138, filed on Oct. 28, 2002, which is a divisional of U.S. application Ser. No. 09/583,902, filed on Jun. 1, 2000, which claims the benefit of U.S. Provisional Application No. 60/137,421 filed on Jun. 4, 1999, which claims the benefit of U.S. Provisional Application No. 60/151,050 filed on Aug. 27, 1999, which claims the benefit of U.S. Provisional Application No. 60/167,681 filed on Nov. 29, 1999, which claims the benefit of U.S. Provisional Application No. 60/174,607 filed on Jan. 5, 2000 and which claims the benefit of U.S. Provisional Application No. 60/182,200 filed on Feb. 14, 2000, the entire disclosures of which are incorporated herein by reference.

### BACKGROUND OF THE INVENTION

1. Field of the Invention

The present invention relates to, e.g., the fields of treating tumors and/or infections of the central nervous system (CNS) and/or other parenchymal organs.

2. Description of the Background

Taurolidine (Bis-(1,1-dioxoperhydro-1,2,4-thiadiazinyl-4)methane) was developed by Geistlich Pharma. It is a white crystalline substance, water soluble up to 2%. It is made up of two molecules of taurinamid and three molecules formaldehyde forming a two-ringed structure bridged by a methylene group.

Taurolidine has primarily an antibiotic and anti-endotoxin effect. It acts by a chemical reaction, so no microorganism resistance has been observed as yet. This effect of taurolidine is mediated by its active metabolites, which are donators of active methylol-groups: Methylol-Taurultam and Methylol-Taurinamide. The active methylol groups inactivate by reacting with the cell wall of bacteria and with the primary amino groups of endotoxins.

Additional effects of taurolidine were reported in the past: inhibition of TNF and IL-1 Beta in mononuclear cells (Bedrosian 1991), inhibition of Tumor Necrosis Factor Toxicity, and inhibition of Peritoneal Tumor Cell Growth in Laparoscopic Surgery (Jacobi 1997).

Taurolidine solutions have been used as instillation or rinsing solutions of the abdominal cavity in cases of peritonitis. In post-operative instillations, conscious patients have reported as a side-effect irritation of the nerves of the peritoneum, and sometimes strong burning sensations which require intravenous administration of pain killers or anaesthesia. Monson et al. PCT International Publication Number WO 92/00743 discloses a selective direct inhibiting effect of Taurolidine and/or Taurultam on certain body tumors. (Monson J R T, Ramsey P S, Donohue J H. Preliminary evidence that taurolidine is anti-neoplastic as well as anti-endotoxin and anti-microbial. Abstract. Br J Surg 77(6) 1990, A711) on B16 melanoma cells and Meth A sarcoma cells in a mice model in vivo, and on fibroblastic tumor cells, LS174T (colon-) carcinoma cells and Jurkat (leukemic-) cells in vitro (International Patent PCT No. PCT/EP91/01269, International Publication Number WO 92/00743 PCT “Use of Taurolidine and/or Taurultam for the treatment of tumors”). However, primary tumors of the brain and medulla of the Central Nervous System (CNS) are very different from those of the body. Nerve cells differ significantly from cells of other organs, and have a much more complex construction. Nerve cells are characterized by a great number of branches which serve to transmit impulses and sensations, including dendrites for reception of impulses, and neurites or axons for emission of impulses. Neurogliae are glia-cells which are present in greater numbers than neurons, and render stability to the nerve cells. Glia-cells are responsible for metabolism and protection of sensitive nerve cells. The cells from which CNS tumors arise have a different metabolism as compared to other tumor cells. Metastases of CNS tumors outside the nervous system are very rare. Effective surgical treatment is often impossible since the tumors are located in functionally important areas, or spread diffusely.

Primary tumors of the brain and spinal cord arise from the different cell types of the CNS. These cell types are neurons, which are responsible for the neuronal function and the glial cells, which have supporting and nutritioning functions. According to the different subtypes of glial and neuronal cells, there are different types of CNS-tumors. The most common brain tumors arise from the glial cells. Various sub-types (astrozytoma, oligodendroglioma, ependymoma, etc.) are encompassed by the term “glioma”.

Gliomas are the most common primary brain tumors. The incidence of gliomas is about 5/100,000 persons per year. More than 50% are glioblastoma, the most malignant form, which is responsible for more than 2.5% of the total tumor associated mortality. More than 95% of the patients die within 2 years following diagnosis despite aggressive therapy including surgery, radiotherapy and chemotherapy.

Brain tumors have some special characteristics as compared to “peripheral” tumors. They act as space occupying lesions, caused by the bony skull. This situation causes herniation and death when the tumor grows larger than can be accommodated. Furthermore, primary brain tumors often metastasize via the cerebrospinal fluid within the whole central nervous system. The brain tumor cells have a lower cohesion within the cell formation as compared to “peripheral” tumor cells (Jänisch W.: Pathologie der Geschwülste des Zentralnervensystems In: Klinische Neuropathologie, J. Cervós-Navarro and R. Ferszt (Eds.) Thieme, Stuttgart, N.Y., 1989). In addition, the metabolism of brain tumors are influenced by the blood/brain barrier.

Both types of tumors, glial and neuronal, can develop malignantly. Malignant gliomas are more frequent as compared to benign gliomas (85% vs. 15%). In the U.S. there are about 20,000 new glioma and medulloblastoma cases per year. The glioblastoma is most common (about 65% among astrocytoma).

Therapeutic options of primary CNS-tumors include surgery, radiotherapy and chemotherapy. Complete resection is often impossible because of poorly defined tumor borders and location within the brain area. Nearly all malignant glioma reoccur within months, 90% on the original site. Reoperation for a recurrent glioma typically extends survival by about 36 weeks (10 weeks with good quality of life). There is no well designed study regarding the beneficial effect of radiotherapy following glioma surgery. In patients older than 65 years, the median survival following tumor biopsy plus radiation is about 17 weeks, and following tumor removal plus radiation about 30 weeks (the peak incidence of glioblastoma is at an age of about 60 years). However, complete tumor removal plus radiatherapy is considered the reference standard in glioma therapy.

Chemotherapy using alkylating agents has a positive response rate of about 30%. A positive response generally extends the survival by 6-8 weeks. However, only about 50% of the patients treated with chemotherapy using alkylating agents are able to maintain regular activities.

Despite progress in diagnosis and treatment, the prognosis of patients with malignant primary CNS-tumors is still poor. The median survival of glioblastoma patients following optimal therapy including complete extirpation and radiation is less than about 10 months (about 1.6 years in grade III astrocytomas). The 1-year survival rate of patients with glioblastoma is about 35%, the 2-year survival rate about 8%.

Some primary malignant central nervous system tumors cannot be treated surgically because of their location or diffuse extension (gliomatosis, diffuse brain stem gliomas). Chemotherapy is not generally recommended, since the response rate on these alcylating agents (BCNU, CCNU, Procarbazine) is about 10% of patients (data from Greenberg M S. Handbook of Neurosurgery. Third edition 1994, Greenberg Graphics Inc., Lakeland, Fla., USA). Heretofore, no therapy could be offered to those patients despite a palliative radiation. Thus, the therapy of primary malignant tumors of the central nervous system has been very unsatisfactory.

In addition, a variety of microdialysis methods and devices are known in other contexts. A number of illustrative methods and devices are shown in the following U.S. patents, the entire disclosure of each of these patents is incorporated herein by reference: U.S. Pat. No. 6,463,312 (entitled “Microdialysis-Probe Integrated with a Si-Chip); U.S. Pat. No. 6,091,976 (entitled “Determination of Glucose Concentration In Tissue”); U.S. Pat. No. 6,030,358 (entitled “Microcatheter and Method for Site Specific Therapy”); U.S. Pat. No. 5,741,284 (entitled “Dialysis Combination and Microdialysis Probe and Insertion Device”); U.S. Pat. No. 5,735,832 (entitled “Reinforced Microdialysis Probe”); U.S. Pat. No. 5,706,806 (involving “an improved linear microdialysis probe assembly [having] a short semipermeable membrane portion containing a flexible, internal support or reinforcement fiber bonded to long lengths of inlet and outlet robing”); U.S. Pat. No. 5,441,481 (involving “a microdialysis probe arranged to have a primary probe, e.g., an electrical probe, secured to it so that the microdialysis probe extends about and is concentric with the primary probe”).

In addition to the foregoing patents, the following U.S. Patents of the present assignee are also incorporated herein by reference as though recited herein in full: U.S. Pat. No. 6,488,912 (entitled “Treatment of Dentoalveolar Infections with Taurolidine And/Or Taurultam”); and U.S. Pat. No. 6,258,797 (entitled “Combating Infection In Delivery Systems”).

There remains a need in the art for new methodologies for, among other things, treating tumors of the central nervous system and/or other parenchymal organs.

### SUMMARY OF THE INVENTION

The present invention relates to a method and application for the treatment and/or prophylaxis of tumors and/or infections in the central nervous system and/or other parenchymal organs in mammalian subjects. In preferred embodiments, a microdialysis device is used to provide an effective dose of taurolidine and/or its metabolites and/or other chemotherapeutic and/or immunotherapeutic and/or antibiotic and/or virostatic and/or fungistatic agents is administered to a mammalian subject suffering from or at risk to growth of tumors of the central nervous system and/or other parenchymal organs.

The preferred embodiments relate to the use of metholyl transfer agents, including Taurolidine and/or Taurultam, for the treatment of tumors of the central nervous system in mammals. Despite the irritation of the nerves of the peritoneum and strong burning sensations which have been side-effects of peritonitis post-operative instillations of Taurolidine, it surprisingly has been found that CNS nerve cells, including the particularly sensitive stem cells of embryo meningeal cells, remain unaffected following administration of Taurolidine/Taurultam solutions.

It was surprising to demonstrate a direct antineoplastic effect of Taurolidine and/or Taurultam on neuronal and glial tumor cell lines. This effect was very unexpected due to the quite different behavior of brain tumor cells as compared to other tumor cells, particularly concerning their response to chemotherapeutic agents. Furthermore, the antineoplastic effect of Taurolidine and/or Taurultam was thought only to be associated with the influence on cell adhesion molecules, which explains the prevention of metastatic tumor growth following endoscopic abdominal tumor surgery. A direct antineoplastic effect on brain tumor cells was very unexpected.

According to one embodiment of the invention, an apparatus for use inserting into a patient so as to provide access to the central nervous system (CNS) or another parenchymal organ for administering a dose of a tumor or infection inhibiting methylol transfer agent to a mammalian subject is provided that includes: an elongated microprobe having a lumen through which a solution can be pumped from a reservoir; the microprobe having a semipermeable region through which a dose of CNS tumor or infection inhibiting methylol transfer agent will pass when the solution is pumped into the microprobe to a region of a tumor and/or infection.

According to another embodiment of the invention, an apparatus for use inserting into a patient so as to provide access to the central nervous system or another parenchymal organ for an agent is provided that includes: a microprobe having a length and a width, the length being substantially longer than the width; the microprobe having a lumen extending lengthwise in the microprobe, the lumen having an opening proximate a distal end of the microprobe; the microprobe having a return path extending from the opening of the lumen to a base end of the microprobe; the microprobe having a semipermeable region around at least a portion of the return path; a reservoir for containing a solution having the agent; a pump for pumping the solution from the reservoir, into the lumen and into the return path; whereby the microprobe can administer the agent through the semipermeable region locally to a region of a tumor and/or infection.

According to another embodiment of the invention, a method is provided that involves a method of treating central nervous system or other parenchymal organ tumors and/or infections comprising inserting an apparatus so as to administer to the central nervous system or other parenchymal organ an agent, the apparatus comprising: a microprobe having a length and a width, the length being substantially longer than the width; the microprobe having a lumen extending lengthwise in the microprobe, the lumen having an opening proximate a distal end of the microprobe; the microprobe having a return path extending from the opening of the lumen to a base end of the microprobe; the microprobe having a semipermeable region around at least a portion of the return path; a reservoir for containing a solution having the agent; a pump for pumping the solution from the reservoir, into the lumen and into the return path; whereby the microprobe can administer the agent through the semipermeable region locally to a region of a tumor and/or infection.

In preferred embodiments, the agent is Taurolidine, Taurultam or a mixture thereof. In some embodiments, the semipermeable region includes a tube bundle. In some embodiments, the semipermeable region includes a semipermeable membrane.

The above and/or other aspects, features and/or advantages of various embodiments will be further appreciated in view of the following description in conjunction with the accompanying figures. Various embodiments can include and/or exclude different aspects, features and/or advantages where applicable. In addition, various embodiments can combine one or more aspect or feature of other embodiments where applicable. The descriptions of aspects, features and/or advantages of particular embodiments should not be construed as limiting other embodiments or the claims.

### BRIEF DESCRIPTION OF THE DRAWINGS

The following figures are shown by way of example and not limitation, in which:

FIG. 1 shows a microprobe formed with a semipermeable membrane according to some illustrative embodiments;

FIG. 2(A) shows a microprobe formed with a semipermeable membrane including a small bundle of tubes according to some illustrative embodiments;

FIG. 2(B) is a cross-sectional view of the microprobe shown in FIG. 2(A) taken along the line 2B-2B shown in FIG. 2(A); and

FIG. 3 shows a microprobe according to some illustrative embodiments implanted within a patient.

### DETAILED DESCRIPTION OF THE PREFERRED EMBODIMENTS

As described above, the preferred embodiments of the present invention relate to a method and application for the treatment and/or prophylaxis of tumors and/or infections in the central nervous system and/or other parenchymal organs in mammalian subjects. In preferred embodiments, a microdialysis device is used to provide an effective dose of taurolidine and/or its metabolites and/or other chemotherapeutic and/or immunotherapeutic and/or antibiotic and/or virostatic and/or fungistatic agents is administered to a mammalian subject suffering from or at risk to growth of tumors of the central nervous system and/or other parenchymal organs.

The Preferred Solutions:

Taurolidine and Taurultam, its intermediate and active metabolite, are methylol transfer agents. They act by transferring methylol groups at the site of action. Both substances have low toxicity and are not cytotoxic against normal cells.

Some preferred embodiments provides for treatment and/or prophylaxis of tumors and/or suppressing of primary and secondary tumors of the central nervous system in mammalian subjects wherein an effective dose of a methylol transfer agent such as Taurolidine and/or Taurultam is administered to a mammalian subject suffering from or at risk of central nervous system tumor growth. Furthermore the invention includes special methods for local application of Taurolidine and/or Taurultam in solution using microdialysis methods, irrigation methods, implantation methods, and angiographic methods. The terms Taurolidine and/or Taurultam as used herein are intended to refer to the compounds Taurolidine, Taurultam, Taurultam-glucose (as described below), and their substantial bioequivalents or agents which act in a substantially similar manner. For example, an aminoglycan derived from Taurultam and any other suitable derivate of Taurolidine and/or Taurultam, or agents which act in a substantially similar manner, can be utilized like Taurolidine and/or Taurultam according to the invention.

The term “treatment” as used herein is intended to refer to treatment, prophylaxis and/or suppression of CNS tumors and/or infections. The preferred embodiments are applicable to treatment of CNS tumors, which may include:

Glioblastoma Multiforme (GBM)

High grade gliomas

Anaplastic oligodendroglioma

Low grade gliomas

Recurrent malignant gliomas

Anaplastic astrocytoma

Advanced metastatic melanoma

Recurrent high grade primary brain tumors

Primary central nervous system lymphoma

Leptomeningeal dissemination of malignant glioma (meningeal gliomatosis).

Treatment takes place primarily in connection with surgical intervention, such as surgical removal of a CNS tumor, as well as postoperative local application of taurolidine and/or Taurultam solution while using, for example, a microdialysis method or an irrigation method. Since the blood/brain barrier is passed by Taurolidine and/or Taurultam, it also may be appropriate to administer 2% taurolidine solutions or 3% Taurultam solutions intravenously through a central catheter. Here, in addition to the antineoplastic action, prevention of infection is also of great advantage for the patient. In this connection, dosage appropriately may be 15-20 g of taurolidine as a 2% solution through a central catheter daily for 7-8 days, or alternatively as 3% Taurultam solution, 20-30 g Taurultam daily, for 7-8 days with adults. This is intended to preserve or improve neurological function and health-related quality of life. For local application in connection with operations in the brain, glucose-based solutions, with or without electrolytes, and which additionally contain 0.2-1% Taurolidine, Taurultam or Taurultam-glucose, are preferred.

Basic treatment solutions preferably are modeled after cerebrospinal solution, contain glucose and electrolytes, are substantially isotonic to the extent possible and have a slightly alkaline pH value of about 7.3-7.35. The following ingredients may be included in a basic solution:

Bicarbonate

Sodium

Potassium

Calcium

Magnesium

Lactate

Chloride

Glucose

Taurolidine, Taurultam, Taurultam-glucose or the like are added to a basic solution.

Exemplary Basic Solution:

A basic solution may, for example, be comprised of Cerebrospinal Fluid (CSF) components as shown in the following table.

| CONSTITUENT | UNITS | CSF | PLASMA | CSF: plasma ratio |
|---|---|---|---|---|
| osmolarity | mOsm/L | 295 | 295 | 1.0 |
| H₂O content | | 99% | 93% | |
| sodium | mEq/L | 138 | 138 | 1.0 |
| potassium | mEq/L | 2.8 | 4.5 | 0.6 |
| chloride | mEq/L | 119 | 102 | 1.2 |
| calcium | mEq/L | 2.1 | 4.8 | 0.4 |
| pCO₂ | mm HG | 47 | 41 | 1.1 |
| pH | | 7.33 | 7.41 | |
| pO₂ | mm Hg | 43 | 104 | 0.4 |
| glucose | mg/dl | 60 | 90 | 0.67 |
| lactate | mEq/L | 1.6 | 1.0 | 1.6 |

Exemplary Amino-sugar/Taurultam-glucose Treatment Agent:

13.6 g Taurultam and 18 g of anhydrous glucose were weighed out into a 250 ml serum bottle, and 200 ml of distilled water were added. The solution obtained was heated to 100° C. for 30 minutes. The clear solution was evaporated in a vacuum until dry. The residue was absorbed in 96% alcohol and placed in an Erlenmeyer flask overnight for forming crystals.

Amino-sugar/Taurultam-glucose crystallized out, and the crystals were suction filtered with a raw yield of 5.3 g.

From alcohol mixed with a few drops of water, white crystals were recrystallized:

Melting point: 168°-170° C.

| | | | | |
|---|---|---|---|---|
| Calculated: | C = 36.23 | H = 6.03 | N = 9.39 | S = 10.74% |
| Found: | C = 36.26 | H = 6.10 | N = 9.09 | S = 10.90% |

The IR spectrum corresponded NMR in DMSO₆ 200 MHZ. Sulfonamide NH coupling to its adjacent CH₂, one OH coupling to CH₂ and three OH's couplings to CH indicated internal loss of water and that the chain had cyclised to form a sugar.

Illustrative Solutions for Use in the Irrigation and/or Microdialysis Methods:

| Solution 1 | 1000 ml contain: |
|---|---|
| Glucose monohydrate for injection purposes | 27.500 g |
| Sodium | 3.382 g |
| Potassium | 0.157 g |
| Ca⁺⁺ | 0.009 g |
| Cl⁻ | 5.520 g |
| Taurultam | 0.5% |

The solution is slightly hypertonic.

The glucose can be replaced by 25 g levulose (fructose).

The solution is then insulin-independent.

| Solution 2 | 1000 ml contain: |
|---|---|
| Sodium | 3.151 g |
| Potassium | 0.156 g |
| Ca⁺⁺ | 0.066 g |
| Mg⁺⁺ | 0.033 g |
| Cl⁻ | 3.900 g |
| Acetate | 2.173 g |
| Taurultam-glucose | 0.5% |

The pH value is set at pH 7.3.

The solutions 1 and 2 are filtered in an appropriately sterile manner with a 0.1 micron sterile filter and aseptically deposited in sterile infusion bottles.

| Solution 3 | 1000 ml contain |
|---|---|
| Glucose monohydrate for injection purposes | 18.330 g |
| Sodium lactate | 2.460 g |
| Sodium chloride | 2.800 g |
| Potassium chloride | 0.187 g |
| Calcium chloride 2 H₂O | 0.147 g |
| Magnesium chloride 6 H₂O | 0.152 g |
| Taurolidine | 1% |

The pH is set at 7.3. The solution is filtered in a sterile manner and aseptically deposited in 100 ml infusion bottles.

| Solution 4 | 1000 ml contain: |
|---|---|
| Sodium chloride | 4.000 g |
| Potassium chloride | 0.050 g |
| Calcium chloride 2 H₂O | 0.066 g |
| Sodium hydrogen carbonate | 0.050 g |
| Taurultam | 1% |

The solution is set at a pH of 7.5 prior to sterilization and subsequently filtered in a sterile manner, deposited in 250 ml infusion bottles and sterilized with steam for 15 minutes at 1210° C.

The Preferred Treatment Methods:

Taurolidine and/or Taurultam may be administered by injection or infusion, or by local application. Isotonic glucose solution and/or artificial cerebrospinal fluid solution as described above may be used containing Taurolidine and/or Taurultam, or a substantial bioequivalent thereof. The local administration can be performed via microdialysis using a probe tube, and/or direct irrigation, and/or implantation of a catheter, and/or single or repeated irrigation. A Microdialysis-method can be utilized in, for example, nonextirpated tumors or reoccurrences as well as in inoperable tumors, e.g., diffuse brain stem gliomas. An irrigation/catheter method may be utilized following complete or incomplete tumor extirpation. In some procedures, one or more of the various methods described herein can potentially be employed together.

Microdialysis Methods:

In some preferred embodiments, an isotonic solution as described above, can be, e.g., stored at body temperature in a tank. A small pump (which can be located, e.g., subcutaneous or outside the body) can force the Taurolidine and/or Taurultam solution via tubular microprobe to the tumor and/or its surrounding region. In some embodiments, the microprobe may be formed of plastic material with a small lumen. The tip of the probe may have a semipermeable membrane so that an osmotic fluid exchange can occur. In this way, the Taurolidine and/or Taurultam can diffuse inside the tumor and its surroundings. Different types of probes can include a probe with a small tip to terminate directly inside the tumor. With large tumors, a large membrane can be provided at the end of the probe to lie inside the tumor cavity or on the surface of the tumor. In some cases, such as with large tumors, more than one probe or a plurality of probes may be implanted or inserted into the patient.

In preferred embodiments, a prepared solution of taurolidine and/or its metabolites and/or other chemotherapeutic and/or immunotherapeutic and/or antibiotic and/or virostatic and/or fungistatic agents is stored in a reservoir. In some embodiments, this is situated out of the body. In some embodiments, it is implanted subcutaneously. Preferably, a small pump (e.g., located external to the body or implanted subcutaneously) forces the solution via microprobes to the tumor and/or infection and/or the surrounding of these processes.

In some preferred embodiments, the microprobes include a semipermeable membrane so that an osmotic fluid exchange can occur. Accordingly, the taurolidine and/or its metabolites and/or other chemotherapeutic and/or immunotherapeutic and/or antibiotic and/or virostatic and/or fungistatic agents can diffuse into the tumor and/or infection and/or the surrounding of these processes.

In some embodiments, different types of microprobes can be provided. For example, some microprobes can have a small tip that ends directly inside the tumor and/or the infection during use. As another example, other microprobes can include a large membrane at or proximate the end that lies inside the tumor cavity and/or the surface of the tumor and/or infection during use. In some treatment or the like processes (such as, e.g., involving larger regions), a plurality of probes can be implanted. In some processes, the plurality of probes used can include similar and/or dissimilar probes depending on circumstances.

In some preferred embodiments, a probe can be made with high-polymer materials as used in known dialysis devices. In some preferred embodiments, a probe can be made with a plurality of small fibres that together form a bundle to reach a particular size.

In some preferred embodiments, the device can enable treatment or the like processes to be performed in which direct and local application of a drug can be achieved without the need for a carrier.

In some preferred embodiments, the device can enable treatment or the like process to be performed in which blood brain barrier is not or is substantially not a problem to pass.

In some preferred embodiments, the device can be selected, varied and/or adapted based on circumstances in that: dependant on the membrane, different molecule sizes are available.

In some preferred embodiments, the device can be substantially self regulating in that, e.g., self regulation of the concentration inside a tumor and/or infection can occur (e.g., inhibiting overdosage).

In some preferred embodiments, the device can achieve a significant reduction in side effects (e.g., to patients).

In some preferred embodiments, the device can achieve a very high local concentration.

In some preferred embodiments, the device can significantly facilitate measurement of concentration.

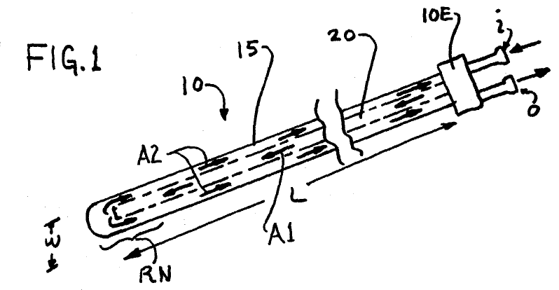

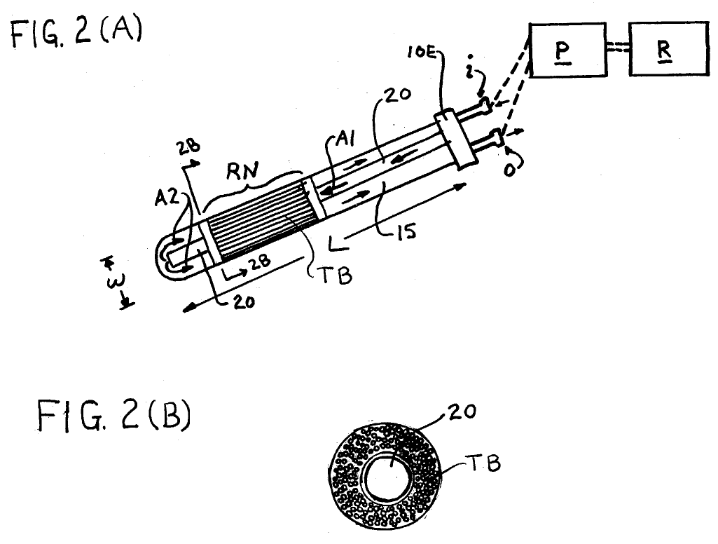

In various embodiments, the various embodiments of microprobes described herein can be adapted and/or modified as would be understood by those in the art based on this disclosure. Additionally, these embodiments and/or alternate embodiments of microprobes can be adapted to include any features, where appropriate, from any of the patents incorporated herein by reference. In some embodiments, microprobes can have a width or diameter (see, e.g., width W shown in FIGS. 1 and 2(A)) of between about 500-1000 μm, while in some embodiments, microprobes can have a width or diameter of between about 200-500 μm, while in some embodiments, microprobes can have a width or diameter of between about 100-200 μm. Other embodiments can have larger or smaller widths or diameters depending on circumstances.

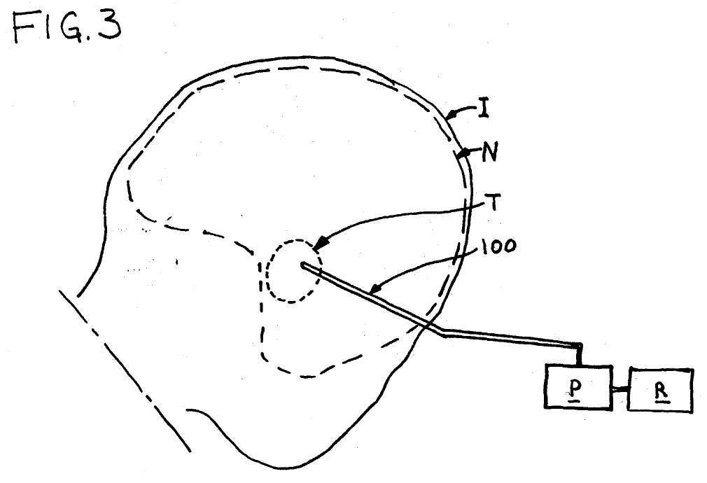

FIG. 1 shows a microdialysis probe 10 according to some illustrative embodiments of the invention. As shown, the probe 10 is preferably an elongated tube-like member with a length L sufficient to extend within a patient (such as, e.g., as shown in FIG. 3) to a desired location and a small width W. Preferably, the probe is a tubular member with a substantially circular cross-section (such as, e.g., as shown in FIG. 3). However, the cross-section can vary in shape depending on circumstances. In preferred embodiments, the probe 10 includes an outer probe delivery region 15 and an internal delivery lumen 20. In some embodiments, the lumen 20 is located substantially along a central axis of the probe and is substantially concentric with the delivery region. However, in other embodiments, the lumen can be situated at other positions or at other locations within or with respect to the probe 10. As shown, by arrows A1, a solution can be delivered via the delivery lumen 20 toward a distal end of the microprobe. Then, as shown by arrows A2 the solution can pass through the delivery region 15 (around the delivery lumen).

As shown, the probe 10 preferably includes a semipermeable region RN through which solution can permeate. In some embodiments, the region RN can be a small region at a tip of the probe 10 (such as, e.g., shown). In some embodiments, the region RN can be set back from the tip of the probe (such as, e.g., shown in FIG. 2). In some embodiments, the region RN can extend a substantial length along the probe. In some illustrative embodiments, the region RN can be less than about 1 mm long, in other embodiments it is between about 1-2 mm long, in other embodiments it can be between about 2-4 mm long, in other embodiments, it can be between about 4-8 mm long, in other embodiments it can be longer than 8 mm long. In some preferred embodiments, the region RN is formed by a semipermeable membrane. Various membranes can be selected by those in the art. Notably, dependent on the membrane, different molecule sizes are available. The microprobes can have a variety of different constructions and sizes, which can vary depending on the particular application. In addition, the lengths L and/or RN, the molecular weight cut-off (which may be, for example, a value less than about 5,000 Daltons, between about 5,000 to 10,000 Daltons, or between about 10,000 to 20,000 Daltons, or between about 20,000 to 40,000 Daltons, or more in various embodiments), and the type of semipermeable membrane used can be selected based on circumstances (such as, e.g., based on characteristics of recovered and/or permeated molecules). Some illustrative semipermeable materials may include one or more of the following materials: cuprophane, polycarbonate, polyethersylfone, polyacrylonitrile; cellulose acetate; regenerated cellulose; and/or various appropriate polymers, hydrophilic membranes, and/or other materials.

In some embodiments, the probes can have a length L of under about 10 cm, while in other embodiments, the length L can be between 10-100 cm, while in other embodiments, the length L can be longer. In some embodiments, the microprobes can be inserted or implanted into a patient using guide cannula or the like. In some embodiments, the microprobes can be substantially rigid, while in other embodiments, the microprobes can be substantially flexible, while in other embodiments, the microprobes can include substantially flexible and/or substantially rigid portions.

In some embodiments, a microdialysis probe provides mass transport in and out of the probe as a function of a concentration gradient across a membrane. Accordingly, the probe can be used to deliver and/or recover compounds from, for example, an extracellular fluid in the local area of implantation surrounding the implanted probe.

FIGS. 2(A) and 2(B) show another illustrative embodiment in which a microprobe includes a semipermeable membrane formed in a region RN. As shown, in this illustrative embodiment, the region RN is set back slightly from a distal tip end of the microprobe. In this illustrative embodiment, the semipermeable membrane is formed by a bundle of small tubes TB.

The bundle of small tubes can include a set of generally parallel tubes all penetrating annular binders or plates at either end of the tube bundle TB. The solution material can be pumped through the tubes in a cross-flow manner (such as, e.g., left to right in FIG. 2(A)). Permeate can leave through the sides of the small tubes. On the other hand, retentate can enter the sides of the small tubes and can pass out of the downstream end of the tubes. Among other things, such a design can enhance membrane/solution contact, etc.

As best shown in FIG. 2(A), in the embodiments shown in FIG. 1 and FIGS. 2(A)-2(B), a small pump P can be provided that pumps solution from a reservoir R into the microprobe at an inlet i. The inlet i can extend to an end member 10E of the microprobe 10 that is configured to direct the solution into the lumen 20. The pump P can also be connected to an outlet o such that solution and/or other fluid can be removed from the microprobe. The outlet o can similarly extend to the end member 10E and the end member can be similarly configured to connect to the region 15 within the microprobe.

FIG. 3 shows an illustrative microdialysis method according to some embodiments in which a prepared isotonic and body-temperature solution is stored in a tank or reservoir R. As shown, a small microdialysis probe or catheter (i.e., microprobe) 100 is implanted inside a patient I (such as, e.g., inserted into the patient's skull and into the patient's central nervous system such as, e.g., within the patient's brain N) toward a tumor and/or infection region T. In the embodiment shown in FIG. 3, the microprobe is implanted inside or within the region T. A small pump P can force the solution via at least one microprobe 100. The microprobe(s) 100 can include any appropriate microprobe, such as shown in patents incorporated herein by reference, in FIGS. 1, 2(A) and/or 2(B), otherwise described herein and/or variations and/or combinations thereof. In some preferred embodiments, the microprobes include a plastic material probe having a small lumen. In some embodiments, the tip of the probe has a semipermeable membrane so that an osmotic fluid exchange can occur. In some embodiments, such as with large tumors, for example, a plurality of probes can be implanted.

In some preferred embodiments, microprobes according to the preferred embodiments can be used for intracerebral infusion with isotonic Taurolin™ solution and/or Taurultam solutions.

Other Methods:

In other embodiments, solutions described herein can be applied using irrigation and/or catheter methods. For example, following the removal of a tumor, or with cystic tumors, direct single or repeated irrigation of the tumor cavity or area may be performed. Furthermore, a catheter can be implanted in the tumor cavity for repeated local administration with Taurolidine and/or Taurultam.

In other embodiments, solutions described herein can be applied using angiographic methods. For instance, another method for regional application of Taurolidine and/or Taurultam may be provided for tumors with blood supply by one or a few dominant feeder arteries. Taurolidine and/or Taurultam may be administered by an angiographic catheter, which may be introduced supraselectively into the feeders. The Taurolidine and/or Taurultam then may be administered once or repeatedly.

In other embodiments, solutions described herein can be applied using implantation methods. For example, following complete or incomplete removal of a tumor, direct single or repeated implantion of a matrix containing Taurolidine and/or Taurultam into the tumor cavity may be performed.

Results:

Taurolidine and/or Taurultam have been found to inhibit directly the growth of CNS tumor cell lines, including neuronal (HT22) as well as glial (C6) tumor cell lines. Furthermore, this action was shown to be selective in that the growth of primary cell lines of a fetal rat central nervous system required significantly higher concentrations and a significantly longer contact time for inhibition, as compared to tumor cells (taking into account a very high general sensitivity of primary cell lines of the fetal rat central nervous system). The effect was concentration-dependent. Antineoplastic effects of concentrations of 0.1 to 4 mg/ml Taurolidine and/or Taurultam in PVP and glucose solution was demonstrated. The tumor cells were inhibited starting after 10 minutes. Following about 1 to 2 hours 90% of the tumor cells were inhibited.

Summary

The tumor-inhibiting agents of the preferred embodiments of the present invention, including Taurolidine and/or Taurultam, may be administered by injection or infusion. Agents in accordance with the present invention may be administered locally using microdialysis utilizing probes, as well as regionally using superselective angiographic catheters with continuous or sequential administration of an agent in accordance with the present invention.

Probes for practicing a microdialysis method in accordance with the invention can be placed using neuronavigation, MRI guidance and/or ultrasound guidance. A diagnostic biopsy can be taken from the tumor to make a histological diagnosis during the same surgical procedure in which treatment utilizing a microdialysis method in accordance with the invention is utilized. Alternatively, during a microdialysis method in accordance with the present invention, fluid can be obtained from the tumor or its surroundings so as to maintain a desired fluid level in the area of the tumor.

An agent in accordance with the present invention can be administered by a permanently or temporarily implanted catheter for continuous or repeated local irrigation of a tumor or its surroundings. The treatment agent can be administered locally by irrigation of the surroundings of a totally or partially extirpated tumor.

In preferred embodiments, Taurolidine and/or Taurultam is administered intravenously in a dosage range of about 50-500 mg/kg per day, sequentially or by continuous administration.

Separately or simultaneously with administration of a methylol transfer agent in accordance with the present invention, other agents can be administered to the patient, including cytotoxic, antineoplastic agents (including alkylating agents, and/or agents involved in tumor metabolism). Alternatively or additionally, if desired, other tumor treating agents may be administered, such as interleukin-1, interleukin-2, interferon, or other immunomodulating agents.

The advantages of combination therapy include:

1) Synergic effects may be realized from employment of a combination therapy with regard to achievement of tumor control and survival improvement.

2) Dosage reduction in administration of antineoplastic medicaments will lead to amelioration of the considerable side effects, such as hair loss, nausea, vomiting, diarrhea, etc.

3) Combination therapy allows for different ways of application of the medicaments, e.g., local Taurolidine/Taurultam administration, systemic general chemotherapy, etc.

Taurolidine and/or Taurultam can be administered by intraperitoneal application in combination with local inthratecal or intravenous general chemotherapy.

This combined administration facilitates prevention of development of metastates and dissemination thereof into the liquor and into the brain during laparotomy or laparoscopic tumor surgery.

### EXAMPLE 1

Taurolidine and Taurultam have been found to inhibit directly the growth of neuronal (HT22, mouse), glial (C6, rat), and mixed neuronaland glial (U373, human) tumor cell lines. For the latter cell line, however, the experiments are not complete as yet. Furthermore, this action was shown to be selective in that the growth of normal central nervous system cells was not significantly inhibited. The effect was concentration-dependent. Antineoplastic effects of concentrations of 0.1 to 4 mg/ml Taurolidine and/or Taurultam was demonstrated. The tumor cells were inhibited selectively beginning after 30 minutes. Following 1 to 3 hours about 90% of the tumor cells were inhibited. For the cell culture, cells were used in RPM1 1640 medium and plated in Falcon flasks. Following incubation with 0.1-4 mg/ml Taurolidine and Taurultam, cytological changes were recorded after 10, 30, 60, 120, 180, 300 minutes, and after 24 and 48 hours.

Beginning following 30 minutes, cytological changes were observed, including: (a) development of vacuoles, and (b) condensation of nuclei, shrinking of cytoplasm, and cell death.

Ultrastructural changes include: swelling of mitochondria, swelling of nuclei, swelling of cytoplasm, and rupture of cell membrane. The first changes occurred after 10 minutes, increasing with time and concentration.

The results of DNA-FACS supported the cytological and ultrastructural observations.

The effect of taurolidine/taurultam on primary CNS-cells was investigated using the brain cells of rat fetuses in a cell culture. We found no significant cytological effect following 48 hours.

For treatment of glioma patients, Taurolidine and/or Taurultam may be administered by injection or infusion, or by local application. The local administration can be performed via (a) microdialysis using tubular probes, and (b) direct irrigation and/or implantation of a temporary or permanent catheter, and single or repeated irrigation.

The Microdialysis-method can be utilized in nonextirpated tumors or reoccurrences as well as in inoperable tumors, e.g., diffuse brain stem gliomas. The irrigation/catheter method may be utilize following complete or incomplete tumor extirpation.

### EXAMPLE 2

### Combined Therapy with Taurolidine and Additionally Antineoplasatic Agents in Patients with Glioblastoma, Gliosarcoma, Anaplasatic Glioma and Astrocytoma

The combination of Taurolidine/Taurultam with antineoplastic agents for treatment of brain tumors such as glioblastoma, astrocytoma and gliosarcoma offers a number of advantages.

The combination of, for example, alkylated agents and Taurolidine and/or Taurultam avoids or reduces side effects such as nausea, vomiting, diarrhea, etc., induced by use of antineoplastic medicaments. The dosage of these antineoplastic medicaments can be reduced by up to half or more and still increase the overall response rate (disease stabilization rate) by synergic effects.

Radiotherapy with its strong side effects can also be avoided or reduced in many cases.

The recurrency rate of dissemination of tumors in primary brain tumors in glioblastoma miltiforma and astrocytoma can also be reduced by a combined therapy.

Of various antineoplastic agents, those medicaments should be chosen which, due to their molecular structure, are unlikely to interact with Taurolidine and/or Taurultam. It is also preferable to direct the combined chemotherapy at the tumor in different ways, e.g., locally to the brain tumor via direct irrigation of Taurolidine and/or Taurultam, or by implantation of a permanent catheter, or via microcialysis in using tubes, and by established chemotherapy i.v. or orally, e.g. by administration of Temozolamide 100 mg/m² once daily for 5 days.

Alternatively, after surgical resection of glioblastoma, localized and sustained delivery of 5-fluorouracil (f-FU) can be provided in combination with Taurolidine and/or Taurultam via central catheter as drop infusion for several days.

In cases of laparoscopic emergency surgery of tumors, laparoscopic cholecystectomy, cholecystitis, laparoscopic cholorectal surgery, etc. in tumor patients as well as in general laparotomy, the intraperitoneal administration of 2% Taurolidine as lavage or instillation in combination with regular i.v. chemotherapy for combating tumors, prevention of metastases and dissemination in the brain, is posssible.

In leptomeningeal dissemination of malignant glioma (meningeal gliomatosis) associated with poor survival intrathecal (IT) chemotherapeutic agents used in combination with local or systemic administration of Taurolidine and/or Taurultam solutions to achieve tumor control and improve survival, may be helpful.

The following antineoplastic agents may be compatible for combination with Taurolidine and/or Tarultam:

| | |
|---|---|
| PCV-Chemotherapy: Combination of: | procarbazine HCl |
| | lomustine (CCNU) (CeeNu) |
| | vincristine sulfate |
| Cisplatin | |
| Methotrexate | |

Cytosinarabinoside (ara-C) cytarabline hydrochlorid

| | |
|---|---|
| Temozolamide | |
| MX2-hydrochloride | |
| Topocetan | |
| Paclitaxel (Taxol) | |
| Interleukin-2 (IL-2) | in simultaneous administration of |
| | Interleukin-1 (IL-1) and |
| | lymphokine-activated killer- |
| | cell or TNF, a combination |
| | with Taurolidine leads to |
| | reduction of toxicity of |
| | the cytokines and is more |
| | agreeable to the patient. |

The nitrosourea medicaments such as ACNU/BCNU/CCNU are generally applied in lower concentration, e.g., 30-50 mg/m² i.v. once per week of 6 weeks. Temozolamide is given orally in a dosage of 50-100 mg/m² for 5 days. MX-2-hydrochlorid is given as antra venous bolus at 20 mg/m² every 28th day for several months until progression occurs.

As another choice, further antineoplastic medicaments are suitable for combination:

| Cyclophospamid | approximately 150 mg/m² |
|---|---|
| Fluorouracil (5-FU) | 40 mg/m² as local bolus |
| or in the form of micropheres as intrathecal (IT) -chemotherapy | |
| Doxorubicin | 10-15 mg/m² i.v. |
| Hydroxycarbamide | |

Cytosinarabinosides (ara-C), thiotriethylene-phosphoramide (thio-TEPA), and Neocarzinostatis can be administered in low doses in IT-chemotherapy in various combinations with Taurolidine and/or Taurultam for improvement of survival and achievement of tumor control and prevention of dissemination, respectively.

Dosage:

The solution for delivery to a patient should contain an effective dosage of Taurolidine and/or Taurultam and/or Taurultam-glucose in the tissue-culture of glioblastoma multiform-tumor cells: as little as 0.1-4 mg/ml Taurolidine inhibits or kills tumor cells in tissue-culture.

Taurultam so far has been shown to be almost twice as effective as Taurolidine, the explanation of which may be found in the equilibrium of Taurolidine in aqueous solution between Methylol-Taurultam and Taurultam.

Taurultam-glucose, on the other hand, has to be dosaged about twice as high as Taurultam, as the molecular weight from Taurultam increases from 136 to 298.

When administered to patients utilizing the irrigation/catheter method described above, a concentration of at least about 4 mg/ml Taurolidine, Taurultam or Taurultam-glucose, respectively, should be utilized.

While illustrative embodiments of the invention have been described herein, the present invention is not limited to the various preferred embodiments described herein, but includes any and all embodiments having modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to examples described in the present specification or during the prosecution of the application, which examples are to be construed as non-exclusive. For example, in the present disclosure, the term “preferably” is non-exclusive and means “preferably, but not limited to.” Means-plus-function or step-plus-function limitations will only be employed where for a specific claim limitation all of the following conditions are present in that limitation: a) “means for” or “step for” is expressly recited; b) a corresponding function is expressly recited; and c) structure, material or acts that support that structure are not recited.

What is claimed is:

1. An apparatus for use inserting into a patient so as to provide access to the central nervous system (CNS) or another parenchymal organ for administering a dose of a tumor or infection inhibiting methylol transfer agent to a mammalian subject, comprising:
an elongated microprobe having a lumen through which a solution can be pumped from a reservoir;
said microprobe having a semipermeable region through which a dose of CNS tumor or infection inhibiting methylol transfer agent will pass when the solution is pumped into said microprobe to a region of a tumor and/or infection.

2. The apparatus of claim 1, further including:
a solution having a methylol transfer agent;
a reservoir containing said solution; and
a pump for pumping said solution into said microprobe via said lumen.

3. The apparatus of claim 1, wherein said microprobe includes a return path around said lumen and said semipermeable region is along said return path.

4. The apparatus of claim 1, wherein said agent is Taurolidine, Taurultam or a mixture thereof.

5. The apparatus of claim 1, wherein said agent is Taurultam-glucose.

6. The apparatus of claim 1, wherein said semipermeable region includes a tube bundle.

7. The apparatus of claim 1, wherein said semipermeable region includes a semipermeable membrane.

8. The apparatus of claim 1, wherein said lumen extends substantially concentrically within said microprobe.

9. The apparatus of claim 8, wherein said lumen extends concentrically beneath said semipermeable region.

10. The apparatus of claim 1, wherein said apparatus is for use inserting into a patient so as to provide access to the central nervous system.

11. The apparatus of claim 1, wherein said apparatus is for use inserting into a patient so as to provide access to a parenchymal organ outside of the central nervous system.

12. An apparatus for use inserting into a patient so as to provide access to the central nervous system or another parenchymal organ for an agent, comprising:
a microprobe having a length and a width, said length being substantially longer than said width;
said microprobe having a lumen extending lengthwise in the microprobe, said lumen having an opening proximate a distal end of the microprobe;
said microprobe having a return path extending from said opening of said lumen to a base end of the microprobe;
said microprobe having a semipermeable region around at least a portion of said return path;
a reservoir for containing a solution having the agent;
a pump for pumping the solution from said reservoir, into said lumen and into said return path;
whereby the microprobe can administer the agent through said semipermeable region locally to a region of a tumor and/or infection.

13. A method of treating central nervous system or other parenchymal organ tumors and/or infections comprising inserting the apparatus of claim 12 so as to administer to the central nervous system or other parenchymal organ an agent, the apparatus comprising:
a microprobe having a length and a width, said length being substantially longer than said width;
said microprobe having a lumen extending lengthwise in the microprobe, said lumen having an opening proximate a distal end of the microprobe;
said microprobe having a return path extending from said opening of said lumen to a base end of the microprobe;
said microprobe having a semipermeable region around at least a portion of said return path;
a reservoir for containing a solution having the agent;
a pump for pumping the solution from said reservoir, into said lumen and into said return path;
whereby the microprobe can administer the agent through said semipermeable region locally to a region of a tumor and/or infection.

14. The method of claim 13, wherein said agent is Taurolidine, Taurultam or a mixture thereof.

15. The method of claim 13, wherein said agent is Taurultam-glucose.

16. The method of claim 13, wherein said semipermeable region includes a tube bundle.

17. The method of claim 13, wherein said semipermeable region includes a semipermeable membrane.

18. The method of claim 13, wherein said lumen extends substantially concentrically within said microprobe.

19. The method of claim 18, wherein said lumen extends concentrically beneath said semipermeable region

20. The method of claim 13, wherein said apparatus is inserted into a patient so as to provide access to the central nervous system.

21. The method of claim 13, wherein said apparatus is inserted into a patient so as to provide access to a parenchymal organ outside of the central nervous system.
